(19) United States
(12) Patent Application Publication
(10) Pub. No.: US 2005/0075670 A1
(43) Pub. Date: Apr. 7, 2005

(54) Signal device with electro-muscle stimulation feature

(76) Inventor: Henrik Bengtsson, Frederiksberg (DK)

Correspondence Address: Reza Green, Esq.;Novo Nordisk Pharmaceuticals, Inc., 100 College Road West, Princeton, NJ, 08540, US

(21) Appl. No.: 10/679,638

(22) Filed: Oct. 6, 2003

Related U.S. Application Data
(60) Provisional application No. 60/419,222.
(60) Provisional application No. 60/428,880, filed on Nov. 25, 2002.

(30) Foreign Application Priority Data
Oct. 7, 2002 (DK) PA 2002 01495

(51) Int. Cl. A61N 1/30

(52) U.S. Cl. 607/3

(57) ABSTRACT

The present invention relates to a signal device for conveniently providing a user with information, comprising a pair of electrodes adapted to be mounted in conductive contact with the skin of a subject, a voltage source for providing a voltage between the pair of electrodes, and control means for controlling the voltage applied between the pair of electrodes, the control means being adapted for identifying a predefined condition or signal and apply a voltage between the pair of electrodes in response thereto. In an exemplary embodiment a drug delivery device is provided further comprising a drug reservoir and expelling means for expelling a drug out of the reservoir through associated outlet means.

### CROSS REFERENCE TO RELATED APPLICATIONS

This application claims priority under 35 U.S.C. 119 of Danish application no. PA 2002 01495 filed Oct. 7, 2002, and U.S. provisional application Nos. 60/419,222 and 60/428,880 filed on Oct. 17, 2002 and Nov. 25, 2002 respectively, the contents of both are fully incorporated herein by reference.

The present invention relates to a signal device for conveniently providing a user with information. The signal device of the invention is suitable for use in combination with drug delivery devices or systems, the signal providing information in the form of a signal or an alarm in respect of a process or an action controlled by, or a state monitored by, a drug delivery device or system.

### BACKGROUND OF THE INVENTION

In the disclosure of the present invention reference is mostly made to the treatment of diabetes by injection of insulin, however, this is only a preferred use of the present invention.

Diabetes mellitus is the common name for at least 2 different diseases, one characterised by immune system mediated specific pancreatic beta cell destruction (insulin dependent diabetes mellitus (IDDM) or type 1 diabetes), and another characterised by decreased insulin sensitivity (insulin resistance) and/or a functional defect in beta cell function (non-insulin dependent diabetes mellitus (NIDDM) or type 2 diabetes).

The principal treatment of type 1 diabetes is straight forward substitution of the missing insulin secretion, whereas treatment of type 2 is more complicated. More specifically, in early stages of type 2 diabetes treatment a number of different types of drugs can be used, e.g. drugs which increase insulin sensitivity (ciglitazones), decrease hepatic glucose output (e.g. metformin), or reduce glucose uptake from the gut (alfa glucosidase inhibitors), as well as drugs which stimulate beta cell activity (e.g. sulfonylurea/meglitinides). However, the above-described deterioration is reflected in the fact that beta cell stimulators will eventually fail to stimulate the cell, and the patient has to be treated with insulin, either as mono therapy, or in combination with oral medication in order to improve glucose control.

Currently, there are two principal modes of daily insulin therapy, the first mode including syringes and insulin injection pens. These devices are simple to use and are relatively low in cost, but they require a needle stick at each injection, typically 3-4 times or more per day. The second mode is infusion pump therapy, which entails the purchase of a portable but relatively expensive pump, for which reason the initial cost of the pump is a barrier to this type of therapy. Although more complex than syringes and pens, the pump offer the advantages of continuous infusion of insulin, precision in dosing and optionally programmable delivery profiles and user actuated bolus infusions in connections with meals. Further, in combination with a blood glucose sensor an infusion pump may provide fully automatic closed loop control of insulin infusion.

Recently less expensive infusion pumps have been proposed which may either be fully disposable providing only the most basic functions such as a constant basal rate, or infusion pump systems comprising a disposable portion in combination with a durable control portion, where the latter may provide many of the more advanced features of the traditional pump.

When using an infusion pump, users desire to hide the pump under clothing so as not to seem different from normal people, however, this is often inconvenient or impractical, especially for diseases such as diabetes, since a user must have access to the external pump for monitoring or administering extra amounts of medication, e.g. bolus infusions during the course of the day in relation to the intake of meals. If a user has concealed the external pump, the user must partially undress or carefully maneuver the external pump to a location that permits access to the display or keypad of the pump.

In order to provide improved access to an externally carried infusion pump, U.S. Pat. No. 4,559,037 discloses a control device for wireless transmission of program instructions to an insulin pump unit which may be either implanted or external to the body. The control device may be used to select a desired basal rate, to select a given infusion schedule or to command the infusion of a bolus having a desired size and infusion profile. The disclosed control device may be programmable and may comprise a display. WO 00/10628 discloses a similar system in which a remote commander can be used to selectively activate a desired function in an external infusion pump device, e.g. delivery of a bolus, selecting a profile for the bolus, or selecting a basal infusion rate. The remote commander comprises a display allowing the user to visually confirm the values entered into the remote commander.

As appears from the above, an infusion pump adapted for or suitable for being carried under the clothing of a user can carry out a large number of actions, e.g. providing pre-programmed infusion rates or profiles, providing user actuated bolus infusions, or providing feed-back controlled closed loop infusion of a desired drug. In addition, most infusion pumps are provided with control means for controlling or checking a number of “internal” conditions of the infusion pump, e.g. flow control means checking that the infused amount of drug corresponds to the set amount, the amount of drug in the reservoir, a low power condition or any other type of malfunction.

For all these types of actions or conditions, it would be desirable to communicate information in respect thereof to the user. Correspondingly, most infusion pumps (especially the more complex of the durable type) is provided with a display capable of displaying all relevant information, e.g. present settings, received instructions, performed user actuated actions, or any type of a variety of alarms. However, as discussed above, this type of infusion pump is preferably worn under the clothing which makes it difficult or inconvenient to check any information displayed directly by the infusion pump.

Addressing this problem, different solutions have been proposed. A basic solution to the problem would be to provide an audible signal or alarm means, e.g. a “beeper” as widely utilized in electronically controlled devices to indicate a given condition. For example, WO 00/10628 discloses a remotely controllable infusion pump device which uses an audible signal to indicate that a given instruction has been received and subsequently that it has been performed.

As discussed above, when using an infusion pump, users often desire to hide the pump so as not to seem different from normal people. Indeed, users would also prefer not to attract attention during use as would often be the case when an audible signal or alarm is sounded. Although it would be possible to set a sound level which is primarily to be heard by the user, it cannot be set too low in order not to be overheard. However, to assure that a given alarm is heard even under relatively noisy conditions, the sound level will in most cases be set so high that it can be heard even under such noisy conditions which again means that the signal or alarm will be considered noisy itself under most normal use conditions, e.g. in closed rooms. Indeed, an alarm may start out at a low level and escalate until acknowledged by the user, however, this would require that the user in most situations would have to manually stop the alarm signal.

WO 00/10628 also discloses that a vibratory means can be used in an infusion pump to indicate a signal or alarm. When provided, such a vibratory means may also be utilized to provide further functions such as generating sufficient vibration to assist in removing gas bubbles from the drug in the reservoir during priming procedures or to agitate the drug in the reservoir in between successive delivery periods. However, to provide such a vibratory alarm is relatively expensive as it normally will have to be implemented as a motor driven imbalance, i.e. as often used in mobile phones, just as it is relatively bulky.

Although the above discussion of signal and alarm means has been based on drug infusion pumps, these considerations would also be applicable to other types of devices such as a body-mounted glucose sensor device.

### DISCLOSURE OF THE INVENTION

Having regard to the above-identified problems and deficiencies, it is an object of the present invention to provide an easy to use and easy to apply signaling means which effectively but discreetly can be used to provide a user with a signal indicative of a given situation, e.g. corresponding to an action controlled or a state monitored by a medical therapy device such as an infusion pump or sensor device, yet can be provided in a cost-effective manner.

More specifically, the present invention is based on the concept that the desired signal function can be based on external electro-muscle stimulation (EMS) in which a conductive pad or electrode is applied externally to the body of a user such that a very weak current can be applied to a muscle or group of muscles to thereby cause them to contract to a degree which is recognisable by the user.

Electro-muscle stimulation (EMS) per se is well known in the medical art and is commonly used in physical or occupational therapy to strengthen atrophied muscles or paralyzed limbs, and also to exercise muscles that are immobilized for long periods of time as a result of muscular or neurological disorders, extended periods of bed rest arising from injury, surgery, or illness. EMS is also useful for the general exercise of functional muscles to improve muscle tone and strength. For example with athletes, EMS can be used to treat muscle injuries as a supplement to conventional conditioning exercises. EMS can also be used to recondition muscles or muscle groups which have, for whatever reason, lost their tone and/or strength, have been injured, or are in need of reconditioning to effect cosmetic improvements.

However, in contrast to the above known applications of EMS, the sole purpose of the present invention is to provide a signal which is recognizable by the user, no therapeutic effect on the involved muscles being intended.

Thus, in a first aspect the present invention provides a fluid (e.g. drug) delivery device comprising a reservoir adapted to contain a liquid drug and comprising, in a situation of use, associated outlet means, as well as expelling means for expelling a drug out of the reservoir through the outlet means. The device further comprises a voltage and energy source and a pair of electrodes adapted to be mounted in conductive contact with the skin of a subject, wherein the control means is adapted for identifying a predefined condition and applying a voltage between the pair of electrodes in response thereto, the flow of current between the pair of electrodes, in a situation of use, resulting in a tactile muscle stimulation.

The outlet means associated with the reservoir may be in direct fluid communication with the reservoir (e.g. in case the expelling means is arranged “before” the reservoir as for a piston pump) or indirect fluid communication (e.g. in case the expelling means is arranged “after” the reservoir as for a membrane pump). The outlet means may be adapted to be brought in fluid communication with infusion means (e.g. a catheter tubing or transcutaneous access means such as an infusion needle, a flexible infusion cannula or a plurality of micro-penetrators) or may comprise these. In the latter case the fluid communication may be established just prior to use, before or after the drug delivery device has been arranged on the user.

The fluid delivery device may be intended to be fully disposable, partially disposable (i.e. with the different components of the device arranged in either a disposable or a durable portion) or durable, it may be prefilled just as it may provide constant rate infusion only or also bolus delivery. The expelling means may be of any desirable nature, such as known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump)), or U.S. Pat. No. 5,527,288 (based on a gas generating pump), which all in the last decades have been proposed for use in inexpensive, primarily disposable drug infusion pumps, the cited documents being incorporated by reference.

The nature of the predefined conditions may be chosen in accordance with the circumstances, e.g. in accordance with the actual configuration of the device (e.g. more or less complex) and the intended use (e.g. providing more or less information). For example, in most applications it would be desirable to provide alarm signals indicative of a “primary” malfunction situation such as when the actual flow rate differs from a preset flow rate, e.g. in case of relative or absolute obstruction of the flow of drug. A pressure sensor may be used to determine if the pressure in the reservoir, expelling means or associated outlet means is above a preset level indicative of blocking. Correspondingly, in most cases it would also be desirable to provide an alarm when the amount of drug in the reservoir is below a preset level, e.g. close to empty. Depending on the nature of the expelling means, the means for detecting a given predefined condition may be provided integrally with means controlling the expelling means or they may be provided as additional control means. For example, in case the expelling means is electronically controlled, values such as the amount of drug remaining in the reservoir may be calculated on the basis of the infusion rate and the initial amount of drug in the reservoir. In case the expelling means is not electronically controlled or it is desirable to provide additional detecting means, such independent detecting means may be in the form of flow sensors or pressure sensors.

Further types of alarms may signal a low power situation or improper operation of the electrodes. For example, the flow of current between the (main) pair of electrodes may be outside a preset range indicating either poor contact or a short circuit. Indeed, in case of very poor contact this information would not be communicated to the user, however, to provide a remedy to this situation it may be desirable to provide information as to the correct operation of the device, e.g. an hourly signal indicating that all monitored conditions are OK. To cope with the situation of electrode malfunction, the device may be provided with two or more electrodes which would be operated by the control means in accordance with the given circumstances to provide the intended signal.

In an exemplary embodiment the drug delivery device is adapted to receive remotely generated commands and to control the drug delivery device in accordance therewith, typically when a user-operated remote control device is used to transmit commands to the drug delivery device. To indicate that a command has been received, the muscle stimulating electrodes may be activated to provide a signal indicative thereof. This signal may be more or less specific, e.g. it may simply indicate that a command has been received, it may indicate that a command from one or more predefined groups of commands has been received, it may indicate that a specific command has been received or, most specifically, the exact nature of a specific command. For example, it may be indicated that a bolus command has been received and subsequently the size (e.g. the number of IU) may be indicated by a corresponding number of current pulses. To provide further feedback information to the user, it may be indicated that a predefined control action has been performed in response to a received command, e.g. a bolus infusion.

The nature of the signals transmitted to the musculature may be chosen in accordance with the intended use and the desired level of sophistication and complexity for the drug delivery device and/or the signal and alarm means.

For example, in a simple implementation, the drug delivery device may be provided merely with an occlusion alarm which provides a stimulating alarm signal having predetermined characteristics in respect of amplitude, polarity, frequency, waveform etc. In other words, one type of signal is intended to provide all users with the desired information. However, as the actual use conditions for a given drug delivery device normally will vary, i.e. the device may be mounted in different locations just as the users may be more or less adipose, it would be desirable if the stimulation characteristics could be adapted to suit the actual conditions, i.e. when placed in a given location on a given user.

Correspondingly, the drug delivery device may advantageously be provided with means allowing the stimulation intensity (or any other stimulation characteristics) to be set by the user. These means may be in the form of user-accessible means accessible directly on the device (e.g. one or more buttons), however, in exemplary embodiments the control means are adapted to receive corresponding commands from external remote control means.

Further, for each type of stimulation signal (e.g. an alarm signal for occlusion or a signal confirming a received command) the corresponding signal may have different pre-set or user-selectable characteristics. An alarm may start out at a low level and escalate until acknowledged by the user, however, this would require that the user in such situations would have to manually stop the alarm signal.

The characteristics of the muscle stimulation signals should be chosen to provide a significant yet pleasant amount of muscle stimulation, e.g. a tickling feel, just as in case different stimulation patterns are used it should be possible to clearly identify the different patterns. The actual voltage supplied between the electrodes, which will ensure the above, will vary in accordance with a number of factors such as individual preferences, adiposity of the user and location of placement. This said, the applied voltage will typically be less than 40V and more typically in the range 3-15V.

In an exemplary embodiment the drug delivery device comprises a mounting surface adapted for application directly to the skin of the user, the pair of electrodes being arranged on the mounting surface which advantageously comprises adhesive means (e.g. a pressure-sensitive adhesive) which allows the device to be affixed to the skin of the subject user.

The electrodes may be of any given type or configuration providing the desired electrical contact under the relevant conditions of use. For example, the electrodes may be of the type described in U.S. Pat. No. 4,522,211 which discloses a surface member defining a well or chamber in which is disposed a porous or reticulated matrix, such as may be provided by a sponge-like plastic-like material, the porous matrix, or “gel pad” as it is often termed, being impregnated with a quantity of electrolytic gel. In the present context the chamber for each provided electrode is advantageously surrounded by adhesive portions of the mounting surface and provided with an easily removable cover arrangement (e.g. a peelable liner) overlying the adhesive and the gel pads to prevent deterioration and leakage of the gel during storage. The specific arrangement, size and configuration of the electrodes will depend on the actual configuration and intended use of the device.

In a second aspect the present invention provides a sensor device comprising a sensor element adapted to be inserted transcutaneously through the skin of a subject and capable of being influenced by a body substance, thereby producing a signal corresponding to a parameter thereof, as well as control means adapted to receive signals from the sensor means and generate command signals in response thereto. The sensor device further comprises a voltage and energy source, and a pair of electrodes adapted to be mounted in conductive contact with the skin of a subject, wherein the control means is adapted for identifying a pre-defined condition on the basis of the command signals and applying a voltage between the pair of electrodes in response thereto, the flow of current between the pair of electrodes, in a situation of use, resulting in muscle stimulation.

In an exemplary embodiment the command signals are in the form of a value indicative of a blood glucose level of the subject. For such an embodiment an alarm signal may be provided when a measured a blood glucose level which is above or below a given range. Further signals may indicate malfunction of a subcutaneously arranged sensor element, that a low voltage condition for the voltage source has occurred or that it is time to change the sensor element.

Turning to the sensor elements per se, relatively small and flexible electrochemical sensors have been developed for subcutaneous placement of sensor electrodes in direct contact with patient blood or other extra-cellular fluid (see for example U.S. Pat. No. 5,482,473), wherein such sensors can be used to obtain periodic or continuous readings over a period of time. This type of sensors are described in, among others, U.S. Pat. Nos. 5,390,671, 5,391,950, 5,568,806 and 5,954,643 which hereby are incorporated by reference.

In a further exemplary embodiment of the invention, a system is provided comprising a sensor portion as well as drug infusion portion, at least one of the portions being provided with muscle stimulating signal means as described above. The system may be in the form of a closed loop system adapted for controlling the blood glucose concentration in the body of a patient, comprising sensor means having a sensor system adapted for providing a sensor signal indicative of a glucose level in blood, the sensor system comprising a sensor element, control means adapted to receive the signals from the sensor system and generate command signals in response thereto in order to keep the blood glucose level of the patient within a desired range, and delivery means for delivering an amount of at least one drug having a blood glucose regulating effect, wherein operation of the delivery means is affected by the command signals.

In a broader aspect, a value indicative of a level of a body fluid parameter is determined, and an effective amount of a drug having a regulating effect on that body fluid parameter is infused into the patient in response to the determined value in order to keep the body fluid parameter level of the patient within a desired range.

The system may be provided as one or more individual units. In an exemplary embodiment a single, self-contained combined sensor and prefilled pump is provided adapted to be mounted on a skin-surface of a user. In a further exemplary embodiment of the system, an individual sensor assembly and an individual drug infusion pump assembly is provided. The two assemblies may be adapted to be locked to each other and utilized as a single unit, or the two assemblies may be mounted on the skin of the user independently but in communication with each, e.g. by cordless communication means. When the system is provided in the form of separate sensor and pump assemblies, it would be possible to offer different types of sensor assemblies and different types of pump assemblies.

Corresponding to a more general aspect of the present invention, a general-purpose signal device is provided comprising a first electrode adapted to be mounted in conductive contact with the skin of a subject, a second electrode adapted to be mounted in conductive contact with the skin of a subject, the first and second electrodes providing a pair of electrodes, a voltage source for providing a voltage between the pair of electrodes, and control means for controlling the voltage applied between the pair of electrodes, the control means being adapted for identifying a predefined condition or signal and apply a voltage between the pair of electrodes in response thereto.

Such a signal device may be incorporated into any skin-mountable device, or it may be provided as a separate skin-mountable signal unit adapted to be in communication with and/or controlled by one or more primary devices. When provided with means for receiving externally generated (cordless) command signals, the signal device may be used in combination with devices or systems which then do not have to be skin mounted. For example, such a signal device may be utilized with a separate infusion pump which may then be carried in a belt or in a pocket. Such an arrangement would provide the user with a choice of signal means, e.g. in some situations it would be acceptable to rely on an audible signal whereas in other situations it would be desirable to use the silent signal means of the present invention.

In a different technical field, people with impaired hearing may use the signal device as a hearing aid, e.g. to help hear the phone ring, an alarm clock sound or any other traditionally audible signal. Indeed, for any given combination of the signal device of the invention and an external device, the two devices will have to be adapted to communicate with each other.

Corresponding to the drug delivery device in accordance with the first aspect of the invention, the sensor device and the general-purpose signal device in accordance with further aspects of the invention may be provided with a “simple” alarm or more advanced versions which can be adapted to suit the actual situation of use, just as for different types of stimulation signals, the corresponding signals may have different pre-set or user-selectable characteristics. Also the electrodes may be configured as described above with reference to the drug delivery device.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject.

### BRIEF DESCRIPTION OF THE DRAWINGS

In the following the invention will be further described with references to the drawings, wherein

FIG. 1 is a schematic representation of a first embodiment of the invention,

FIG. 2 is a schematic representation of a second embodiment of the invention,

FIG. 3 shows a third embodiment,

FIG. 4 shows a fourth embodiment in a side view, and

FIG. 5 shows a view of the mounting surface of the fourth embodiment.

### DESCRIPTION OF EXEMPLARY EMBODIMENTS

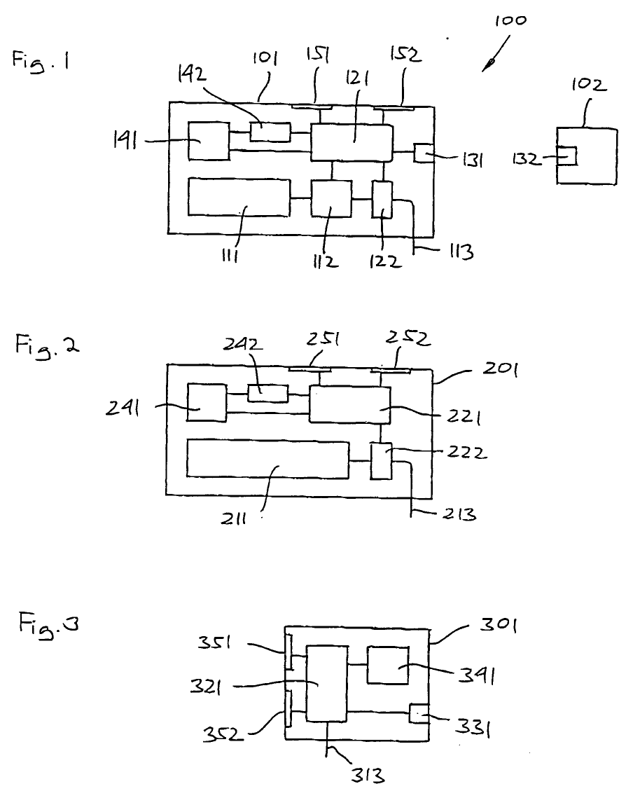

FIGS. 1-5 show schematic representations of embodiments of the invention. Correspondingly, the configuration of the different structures as well as there relative dimensions and positions are intended to serve illustrative purposes only.

More specifically, FIG. 1 shows a drug infusion system 100 comprising a drug delivery device 101 and an optional remote control device 102. The drug delivery device comprises a drug reservoir 111 in fluid communication with a pump 112 (e.g. a membrane pump) adapted for infusing a drug into a body of a user via infusion needle 113 in accordance with instructions received from the control means in form of a micro processor 121. The pump may be of the metering type, i.e. the amount of drug infused corresponds to the controlling signals received from the processor or the infusion unit may be provided with detecting means for determining the amount of drug actually infused. In the shown embodiment a separate flow monitor and occlusion detector 122 is provided downstream of the pump in communication with the processor. A voltage and energy source 141 is provided in the form of a battery supplying energy to the processor as well as the pump and detecting means (via the processor). A typical voltage supplied by the battery would be 1.5 or 3V, however, to provide a muscle stimulating current it is necessary to transform the battery voltage to a higher level using e.g. a switch mode power supply (SMPS) 142 transforming the voltage to for example 20V. Although the SMPS is shown as a separate element, it is preferably provided integrally with a processor unit.

The drug delivery device further comprises a pair of electrodes 151, 152 adapted to be mounted in conductive contact with the skin of a subject. In the shown schematic representation the electrodes and the infusion needle are arranged on different sides of the device, however, for an actual implementation of the invention, the drug delivery device advantageously comprises a mounting surface adapted for application to the skin of a subject, the pair of electrodes as well as the infusion needle being arranged on the mounting surface. This consideration also applies to the second and third embodiments.

The processor is programmed (being either pre-programmed or programmable) for identifying one or more predefined conditions and applying a voltage between the pair of electrodes in response thereto, such that the flow of current between the pair of electrodes will result in muscle stimulation when the electrodes are in conductive contact with the skin of the user. For example, an alarm may be initiated in case an occlusion is detected by the occlusion detector 122.

In the shown embodiment the processor 121 is associated with a receiver 131 for receiving user-controllable command signals from a remote control device 102 comprising a corresponding transmitter 132. The remote device may be used to set the characteristics for the (alarm) signals transmitted via the electrodes as described in detail above, however, the remote device is preferably in the form of a general command unit by which the unit can control the drug delivery device, e.g. setting an infusion rate, program a bolus infusion of a desired size. The communication is preferably cordless based on e.g. RF or IR transmission.

The first embodiment is based on one-way transmission of commands from the remote device to the drug delivery device, however, the transmission and receiving means may be adapted to also transmit information from the infusion to the remote device, e.g. the actual infusion rate or the remaining amount of drug in the reservoir, the remote device being provided with a display for displaying such information.

FIG. 2 shows a schematic representation of a second embodiment of the invention, in which like numerals are used to identify like structures. The drug delivery device 201 of the second embodiment corresponds generally to the first embodiment, however, the reservoir and pump means are provided by a combined infusion unit 211 operating independently with regard to the control processor 221 merely providing a basal rate infusion of a drug. Such an infusion unit may be based on an osmotic pump, a bleeding hole pump or a gas generating pump. In the shown embodiment a separate flow monitor and occlusion detector 222 is provided downstream of the pump in communication with the processor. The second embodiment represents a simpler drug delivery device comprising no receiving means, however, means for programming the processor 221 may be provided on the drug delivery device (not shown).

FIG. 3 shows a schematic representation of a third embodiment of the invention, in which like numerals are used to identify like structures. The third embodiment is in the form of a sensor device 301 comprising a needle-formed sensor 313 adapted to be inserted subcutaneously through the skin of a subject and capable of being influenced by a body substance and producing a signal corresponding thereto. A battery 341 for energizing the processor is provided, the processor comprising a SMPS to boost the voltage from the battery.

The processor 321 is adapted to receive signals from the sensor means and generate command signals in response thereto. The command signals may be transmitted to an external device via transmission means 321, for example to an infusion pump device of the type corresponding to the first embodiment. Preferably the system is in the form of a closed loop system adapted for controlling the blood glucose concentration in the body of a patient, comprising sensor means having a sensor system adapted for providing a sensor signal indicative of a glucose level in blood, the sensor system comprising a sensor element, control means adapted to receive the signals from the sensor system and generate command signals in response thereto in order to keep the blood glucose level of the patient within a desired range, and delivery means for delivering an amount of at least one drug having a blood glucose regulating effect, wherein operation of the delivery means is affected by the command signals.

The sensor device further comprises a pair of electrodes 351, 352 adapted to be mounted in conductive contact with the skin of a subject. Corresponding to the invention, the processor is programmed to identify one or more predefined conditions and apply a voltage between the pair of electrodes in response thereto, such that the flow of current between the pair of electrodes will result in a muscle stimulation signal when the electrodes are in conductive contact with the skin of the user. For example, an alarm signal may be initiated in case the measured blood glucose level is outside a predefined range.

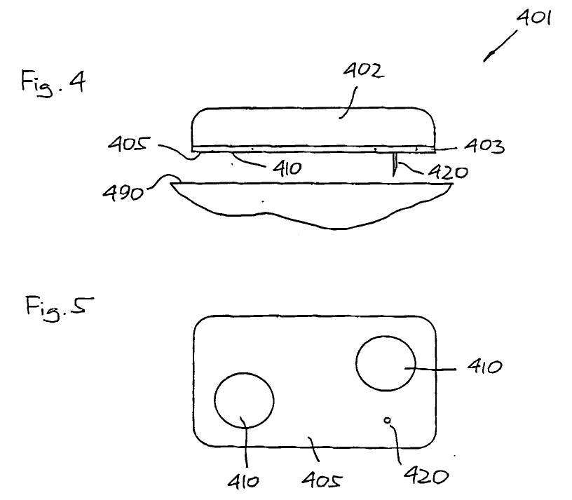

FIG. 4 shows a skin-mountable device 401 which may represent a drug delivery device as well as a sensor device. The device comprises a housing 402 with a base plate portion 403 having a lower mounting surface 405 comprising a pressure-sensitive adhesive which allows the device to be affixed to a skin surface 490 of a subject user. The base plate portion is provided with two well-formed recesses in which “gel pads” 410 impregnated with a quantity of conductive electrolytic gel are arranged serving as electrodes. Protruding from the mounting surface is arranged a needle-formed device 420 which may represent an infusion needle or a sensor element. When supplied to the user, the mounting surface is provided with an easily removable liner (not shown) overlying the adhesive and the gel pads. Correspondingly, the needle device is also protected by a cover (not shown) or the device is supplied with the needle device in a retracted position.

In FIG. 5 the mounting surface provided with two electrodes 410 and a needle device is shown. The specific arrangement, size and configuration of the electrodes as well as the position of the needle device relative thereto is only illustrative and may be adapted in accordance with the actual configuration and intended use of the device.

In the above description of the exemplary embodiments, the different structures providing the desired relations between the different components just as the means providing the describer functionality for the different components (processor means, transmitting and receiving means, memory and timer means) have been described to a degree to which the concept of the present invention will be apparent to the skilled reader. The detailed construction and specification for the different structures are considered the object of a normal design procedure performed by the skilled person along the lines set out in the present specification.

1. A fluid delivery device (101) comprising:
a reservoir (111) adapted to contain a fluid and comprising, in a situation of use, associated outlet means (113),
expelling means (112) for expelling a fluid out of the reservoir through the outlet means,
a voltage source (141),
a pair of electrodes (151, 152) adapted to be mounted in conductive contact with the skin of a subject,
control means (121) adapted for identifying a predefined condition and applying a voltage between the pair of electrodes in response thereto, the flow of current between the pair of electrodes, in a situation of use, resulting in muscle stimulation.

2. A fluid delivery device as defined in claim 1, wherein the predefined condition belongs to the group of conditions comprising:
an actual fluid delivery rate which differs from a preset fluid delivery rate,
a pressure in the reservoir, expelling means or associated outlet means above a preset level,
an amount of fluid in the reservoir below a preset level,
a flow of current between the pair of electrodes outside a preset range,
a low voltage condition for the voltage source, and
a preset timer interval.

3. A fluid delivery device as defined in claim 1 or 2, wherein the control means is adapted to receive remotely generated commands and to control the fluid delivery device in accordance therewith, the predefined condition belonging to the group of conditions comprising:
receiving a command from a predefined groups of commands,
receiving a predefined command, and
performing a predefined control action in response to a received command.

4. A fluid delivery device as defined in claim 1, further comprising a mounting surface (405) adapted for application to the skin of a subject, the pair of electrodes (410) being arranged on the mounting surface.

5. A fluid delivery device as defined in claim 4, wherein the mounting surface comprises adhesive means which allows the device to be affixed to the skin of the subject user.

6. A fluid delivery device as defined in any of the previous claims, wherein the outlet means comprises a hollow infusion needle (113, 420) communicating, in a situation of use, with the interior of the reservoir.

7. A fluid delivery device as defined in claim 5, wherein the infusion needle comprises a distal pointed end (61) adapted to penetrate the skin of the subject, the infusion needle being moveable between a first position in which the pointed end of the needle is arranged in a retracted position relative to the mounting surface, and a second position in which the pointed end of the needle projects from the mounting surface.

8. A sensor device (301) comprising:
a sensor means (313) adapted to be inserted transcutaneously through the skin of a subject and capable of being influenced by a body substance and producing a signal corresponding thereto,
control means (321) adapted to receive signals from the sensor means and generate command signals in response thereto,
a voltage source (341),
a pair of electrodes (351, 352) adapted to be mounted in conductive contact with the skin of a subject,
wherein the control means is adapted for identifying a predefined condition on the basis of the command signals and applying a voltage between the pair of electrodes in response thereto, the flow of current between the pair of electrodes, in a situation of use, resulting in muscle stimulation.

9. A sensor device as defined in claim 8, wherein the command signals are in the form of a value indicative of a blood glucose level of the subject, and wherein the predefined condition belongs to the group of conditions comprising:
a blood glucose level which is outside a given range,
a signal from the sensor means is outside a given range,
a low voltage condition for the voltage source, and
a preset timer interval.

10. A sensor device as defined in claim 8, further comprising a mounting surface adapted for application to the skin of a subject, the pair of electrodes being arranged on the mounting surface, the mounting surface preferably comprising a pressure-sensitive adhesive which allows the device to be affixed to the skin of the subject user.

11. A signal device comprising:
a first electrode adapted to be mounted in conductive contact with the skin of a subject,
a second electrode adapted to be mounted in conductive contact with the skin of a subject, the first and second electrodes providing a pair of electrodes,
a voltage source for providing a voltage between the pair of electrodes,
control means for controlling the voltage applied between the pair of electrodes, the control means being adapted for identifying a predefined condition or a signal and apply a voltage between the pair of electrodes in response thereto.

12. A signal device as defined in claim 11, further comprising a mounting surface adapted for application to the skin of a subject, the pair of electrodes being arranged on the mounting surface, the mounting surface preferably comprising adhesive means which allows the device to be affixed to the skin of the subject user.

13. A signal device as defined in claim 11, wherein the control means is adapted to receive remotely generated commands and to apply a voltage between the pair of electrodes in response thereto.
